(19) United States
(12) Patent Application Publication
(10) Pub. No.: US 2024/0331879 A1
(43) Pub. Date: Oct. 3, 2024

(54) AUTOMATED ALERTING SYSTEM FOR RELEVANT EXAMINATIONS

(71) Applicant: KONINKLIJKE PHILIPS N.V., EINDHOVEN (NL)

(72) Inventors: Vadiraj Krishnamurthy HOMBAL, WAKEFIELD, MA (US); Thusitha Dananjaya De Silva MABOTUWANA, REDMOND, WA (US)

(21) Appl. No.: 18/293,414

(22) PCT Filed: Jul. 15, 2022
(86) PCT No.: PCT/EP2022/069926

§ 371 Date: Jan. 30, 2024

Related U.S. Application Data
(60) Provisional application No. 63/228,178, filed on Aug. 2, 2021.

(51) Int. Cl. G16H 50/70; G16H 10/40; G16H 10/60

(52) U.S. Cl. CPC G16H 50/70; G16H 10/40; G16H 10/60

(57) ABSTRACT

A method for providing feedback to a radiologist, including: receiving a plurality of medical reports; processing the plurality of medical reports to produce a processed report that extracts patient medical information; receiving a feedback request related to a radiology report; identifying a medical report related to the feedback request; and providing a to notification the radiologist regarding the identified medical report related to the feedback request.

### TECHNICAL FIELD

Various exemplary embodiments disclosed herein relate generally to an automated alerting system and method for relevant examinations.

### BACKGROUND

Radiology plays a critical role in the identification of patient disease. Radiologists encounter patients cases of varying complexity and often arrive at their diagnoses under uncertainty due to the complexity of the case, missing patient history, or poor image quality. This can lead to diagnostic errors, diagnostic uncertainty, and potentially delay patient diagnoses resulting in adverse effects on patient health.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for providing feedback to a radiologist, including: receiving a plurality of medical reports: processing the plurality of medical reports to produce a processed report that extracts patient medical information: receiving a feedback request related to a radiology report: identifying a medical report related to the feedback request; and providing a notification to the radiologist regarding the identified medical report related to the feedback request.

Various embodiments are described, further comprises visualizing the identified medical report and the radiology report for the radiologist.

Various embodiments are described, wherein the feedback request is a request from the radiologist to provide feedback related to the radiology report.

Various embodiments are described, wherein the feedback request is extracted from the radiology report using a language processing model.

Various embodiments are described, further comprising determining the relevance of the plurality of medical reports to one another and wherein identifying a medical report related to the feedback request is based upon the determined relevance of the plurality of medical reports to one another.

Various embodiments are described, further comprising receiving an acceptance/rejection response from the radiologist and updating a model configured to identify a medical report related to the feedback request.

Various embodiments are described, wherein identifying a medical report related to the feedback request includes calculating a score based upon anatomy identified in the radiology report, reference to the radiology report in a subsequent examination, and an indication that a subsequent examination is a follow-up of a recommendation in the radiology report.

Various embodiments are described, wherein identifying a medical report related to the feedback request is based upon a machine learning model or a rules based model.

Various embodiments are described, further including: estimating the similarity between the plurality of medical reports based upon the extracted patient medical information: clustering similar medical reports; and inferring a group type for the clustered medical reports and labeling the clustered medical reports with the inferred group type.

Various embodiments are described, wherein processing the plurality of patient medical reports includes document structure processing, syntactic parsing of an output of the document structure processing, extracting entities from an output of the syntactic parsing, and determining an anatomy inference on the extracted entities.

Various embodiments are described, wherein the plurality of medical reports include radiology reports and pathology reports.

Further various embodiments relate to a device for providing feedback to a radiologist, including: a memory: a processor coupled to the memory, wherein the processor is further configured to: receive a plurality of medical reports: process the plurality of medical reports to produce a processed report that extracts patient medical information: receive a feedback request related to a radiology report: identify a medical report related to the feedback request; and provide a notification to the radiologist regarding the identified medical report related to the feedback request.

Various embodiments are described, further comprises visualizing the identified medical report and the radiology report for the radiologist.

Various embodiments are described, wherein the feedback request is a request from the radiologist to provide feedback related to the radiology report.

Various embodiments are described, wherein the feedback request is extracted from the radiology report using a language processing model.

Various embodiments are described, further comprising determining the relevance of the plurality of medical reports to one another and wherein identifying a medical report related to the feedback request is based upon the determined relevance of the plurality of medical reports to one another.

Various embodiments are described, further comprising receiving an acceptance/rejection response from the radiologist and updating a model configured to identify a medical report related to the feedback request.

Various embodiments are described, wherein identifying a medical report related to the feedback request includes calculating a score based upon one of anatomy identified in the radiology report, reference to the radiology report in a subsequent examination, and an indication that a subsequent examination is a follow-up of a recommendation in the radiology report.

Various embodiments are described, wherein identifying a medical report related to the feedback request is based upon a machine learning model or a rules based model.

Various embodiments are described, further including: estimating the similarity between the plurality of medical reports based upon the extracted patient medical information: clustering similar medical reports; and inferring a group type for the clustered medical reports and labeling the clustered medical reports with the inferred group type.

Various embodiments are described, wherein processing the plurality of patient medical reports includes document structure processing, syntactic parsing of an output of the document structure processing, extracting entities from an output of the syntactic parsing, and determining an anatomy inference on the extracted entities.

Various embodiments are described, wherein the plurality of medical reports include radiology reports and pathology reports.

### BRIEF DESCRIPTION OF THE DRAWINGS

In order to better understand various exemplary embodiments, reference is made to the accompanying drawings, wherein:

FIG. 1 illustrates the clinical flow associated with the detection, diagnosis, treatment, and monitoring of patient disease;

FIG. 2 illustrates the various systems that make up the radiology feedback system and the flow of data between the different systems:

FIG. 3 illustrates a report processing pipeline of the radiology and pathology reports carried out by the automated report processing system and the anatomy inference system; and

FIG. 4 illustrates an exemplary hardware device for implementing the radiology feedback system.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

In current radiology workflows there is a certain amount of imbalance in information sharing between radiology and pathology. After evaluating medical images, a radiologist may recommend a biopsy. The biopsy may be taken by an interventional radiologist and then a pathologist evaluates the biopsy report, but the radiologist does not necessarily know what the diagnosis from pathology is. This is important because a radiologist looks at the anatomy level in a radiological report, whereas the pathologist gains a cellular view of the same disease from the pathology report.

In order to improve their practice, radiologists must seek and receive feedback from subsequent patient exams and evaluate the accuracy and relevance of their original diagnoses. Some examples of instances where a radiologist can obtain feedback from a subsequent examination are as follows. A radiologist reading a complicated magnetic resonance image (MRI) who recommended a biopsy receives the subsequent relevant pathology report so that the radiology and pathology outcomes may be compared. A radiologist reading a positron emission tomography (PET) scan with an uncertain nodule diagnosis may then receive a subsequent computerized tomography (CT) scan to confirm the presence of the nodule. A radiologist recommending a follow-up CT scan receives the follow-up examination to track patient health.

It is critical for the radiologist to get feedback of their diagnosis so that they can improve their practice. In some cases, for example, a radiologist may refer to a disease in a certain part of the anatomy, but the tissue samples extracted during the biopsy procedure may fail to capture the malignant tissues even though the general anatomy is the same. As a result, the pathologist may not see anything wrong. This is a well-known problem in medical practice. Moreover, feedback and information sharing between radiologists and pathologists is important for improving the quality of patient diagnosis and alleviating the problems identified above.

Effective feedback helps radiologists improve their diagnosis skills and reduces errors. Additionally, effective feedback as described herein reduces the number of unnecessary biopsies, which are invasive and may entail significant consequences and potential side effects. It is desirable to minimize the number of biopsies while still performing biopsies on the right anatomy, at the right location, and making sure that only those patients who need biopsies are biopsied. Radiologists need to be careful to not request too many biopsies as doing so may lead to many unnecessary biopsies.

It has been observed from analyzing radiology and pathology data that there are cases where there have been over prescriptions of biopsies and under prescription of biopsies. One embodiment of a radiology feedback system described herein is able to inform the radiologist that a pathology report has been generated that is related to a previous radiology report generated by the same radiologist. The radiologist may then review the pathology report to determine if their diagnosis was correct or not.

A systematic institution-wide framework is needed to support such feedback so that radiologists may strive for continuous improvement. Key sub-systems are described herein that may be utilized for such a framework. Specifically, a feedback system is described wherein a radiologist may request feedback on a radiology examination that he or she is currently reading. The feedback system monitors all relevant channels and repositories and subsequently alerts, via various channels, the requesting radiologist when the relevant examination is detected and presents them with content of the examination.

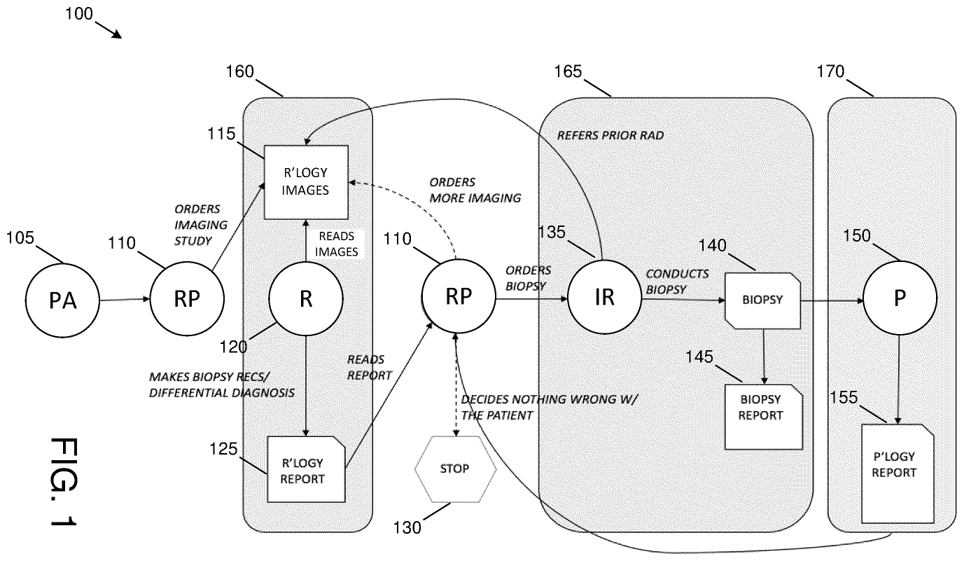

FIG. 1 illustrates a typical clinical flow 100 associated with detection, diagnosis, treatment, and monitoring of patient disease. The clinical flow 100 associated with the detection, diagnosis, treatment, and monitoring of patient disease involves several stakeholders such as the patient 105, referring physician 110, radiologist 120, interventional radiologist 135, pathologist 150, etc. The clinical flow 100 is made up of complex information flows with communication between stakeholders. However, as seen in FIG. 1, while the radiologists play a key role in this flow, they receive little, if any, feedback on their efforts from other stakeholders who are the consumers of their output.

A patient 105 goes to see their physician 110 with a health problem. This physician or referring physician 110 orders an imaging study. The imaging study is carried out in a radiology department 160. The radiologist 120 reviews the imaging study and produces a radiology report 125. The radiologist 120 may recommend a biopsy based upon the radiology images 115 in the radiology report 125. The referring physician 110 receives and reviews the radiology report 125. If the referring physician determines that there is nothing wrong with the patient, then the clinical flow 100 ends 130. If the radiology report does not provide a clear indication of the patient's diagnosis, the referring physician 110 may order that further imaging is needed. In other situations, the radiology report may indicate that a biopsy is required to further diagnose the patient. For example, lesions may be visible in the imaging study that would require a biopsy to fully understand the nature of the lesions.

The interventional radiologist 135 (or other medical professional) may then conduct the biopsy to extract tissue sample(s) 140 to be analyzed by the lab to produce a biopsy report 145. The pathologist 150 then reviews the biopsy results and report 145 and produces a pathology report 155. The pathology report 155 is then sent back to the referring physician 110 for further diagnosis and treatment of the patient 105.

As can be seen in the clinical flow 100, the radiologist 120 does not receive feedback from the pathology report 155 that would allow the radiologist to receive feedback regarding the accuracy of their evaluation of the imaging study and their recommendation. One important reason for such a gap in the clinical flow 100 is that clinical data related to patients is spread across multiple silos and often it is the referring physician that acts as a data aggregator. These silos may include a radiology department 160, an interventional radiology department 165, and a pathology department 170. As a result, gathering of relevant patient context in several clinical settings is typically manual. Consequently, monitoring and processing systems are not standardized. The radiology feedback system described herein relies on the capability to gather data from several sources in a hospital system.

Another significant reason for a lack of radiologist feedback is that patients have complex clinical histories with multiple radiology and lab exams with multiple chronic pathologies (e.g., a female patient with hip fracture, breast cancer, incidental lung nodules). As a consequence, it is not trivial to create this feedback system without overloading the requesting radiologist with information. In an efficient system, only the relevant subsequent exams are provided to the requesting radiologist. As described herein, a radiology feedback system may include an information processing system that analyzes examination content to identify the correct subsequent examination that is relevant to the requesting radiologist.

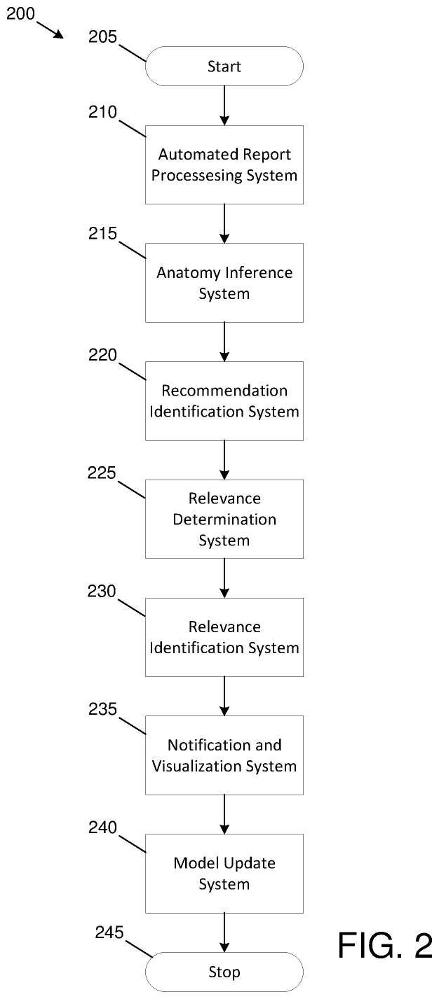

FIG. 2 illustrates the various systems that make up the radiology feedback system 200 and the flow of data between the different systems. The radiology feedback system 200 includes an automated report processing system 210, anatomy inference system 215, recommendation identification system 220, relevance determination system 225, relevance identification system 230, notification visualization system 235, and model update system 240. The radiology feedback system 200 provides a solution to classify patient radiology and pathology exams according to anatomy of disease/pathology enabling gathering and presentation of relevant data and presentation in different disease contexts.

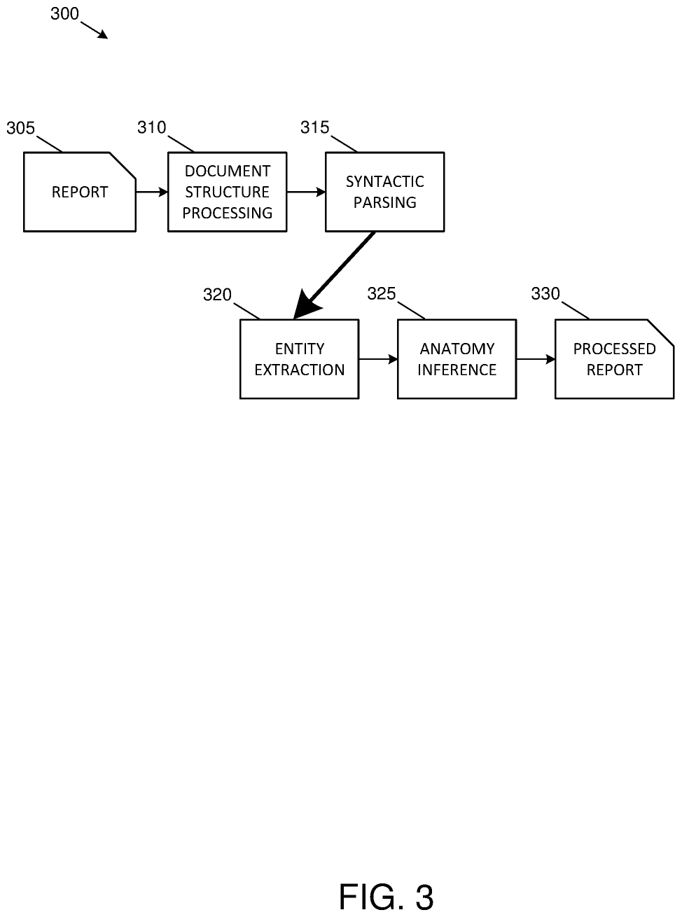

In FIG. 2, the automated report processing system 210 and anatomy inference system 215 analyze all of the patient reports in the radiology department 160 and the pathology department 170 and these processed reports are input into the recommendation identification system 220. FIG. 3 illustrates a report processing pipeline 300 of the radiology and pathology reports carried out by the automated report processing system 210 and the anatomy inference system 215. All pathology and radiology reports are first individually passed through the processing pipeline 300.

The first step of the report processing pipeline 300 is a document structure processing 310 that identifies document structure such as sections, headers, paragraphs, sentences, etc. The document structure processing 310 receives a patient report 305 and analyzes the structure of the report. Because certain parts of the reports may have the most relevant information used to identify similarities, these parts of the reports may be identified. For example, the diagnosis sections of pathology reports and X-ray reports may be a good source to identify similarities. The document structure processing 310 may include a section typing model that determines different sections of the record and then determines the types of each of the sections identified. This may be done using machine learning or rule based models. In one example, a rule based model may be used to segregate the document based upon vertical spacing. A rule based model that uses vertical spacing in the reports has been developed and shown to be robust and configurable. For example, at a specific medical site or system, a specific report structure is used. Knowledge of this report structure may be used to develop the section typing model. The output of the model will be sections of the document and an associated type of the section. The text in the sections may then be further processed to identify paragraphs and sentences.

The document structure processing outputs are then fed into the syntactic parsing 315. The syntactic parsing 315 may include language models that process specific text in the document sections. For example, sentences may be syntactically parsed to identify noun phrases. The language model may include a model that parses the text and converts phrases to a vector space that indicates the meaning of the phrases. That is phrases with the same meaning would be mapped to the same vector. The text may be broken into noun phrases, and then within the noun phrases the word vectors are aggregated. The identified noun phrases are then processed by the entity extraction module 320 to, for example, identify anatomical regions (e.g., lower left lobe, gall bladder, etc.), findings/diagnoses (e.g., pulmonary nodule, cirrhosis, hepatocellular carcinoma, etc.), and procedures (e.g., salpingostomy, colonoscopy etc.).

The anatomy inference module 325 then provides clinical ontology-based anatomy labels to the extracted entities (anatomies, findings/diagnoses, and procedures) based on examination meta data, document structure, and paragraph, and sentence level information (e.g., pneumothorax→lung, salpingectomy→fallopian tube, CT chest LLU→lung). The examination meta-data, document structure, the syntactic parse of the sentences, the extracted entities, their labels, and the relationships between these are all saved in a processed report 330. The anatomy inference system 215 helps to establish similarities between the examinations.

The recommendation identification system 220 identifies recommendations in radiology reports and establishes the relevance of subsequent exams to the recommendations. For example, a radiologist may recommend a biopsy based upon an imaging study because of a highly suspicious lesion. In another situation, the radiologist may recommend a new CT scan in three months to further monitor the progress of an already diagnosed disease. In other situations, further imaging studies may be requested in order to get more information for diagnosis. The recommendation identification system 220 may include a multiclass classifier that determines the type of recommendation, e.g., no recommendation, biopsy, further imaging required now, schedule a follow up imaging study, etc. Any type of machine learning classifier or rules based classifier may be used.

Once a recommendation is identified, the recommendation identification system will identify the sentence(s) specifying the recommendation. Based upon these sentences, the following are determined: what needs to be done; when does it need to be done; how does it need to be done; and where does it need to be done (e.g., what specific anatomy needs to be imaged). The “where” may be very important. For example, if the lung is identified, further clarification that it is the lower left lobe of the lung where a nodule has been detected may be determined. This recommendation may indicate further radiology examination or a biopsy for a pathological examination. Further, when a radiologist evaluates an imaging study, they may identify prior imaging or pathology examinations that were consulted and considered. These prior examinations will be noted as relevant to the examination report being processed.

A further request for feedback may be input by the radiologist at this point to request feedback regarding the recommendation. This may be done using a requesting interface (RI). The RI collects the request of a radiologist interested in receiving feedback for an examination. The request collected using RI may include the following information:
Examination Data: Accession, medical record number (MRN), Date
Request Data: Radiologist ID, Request Date
Reason for Request: finding(s), diagnosis, recommendation(s) to be tracked.
   In addition, provision is made for the requesting radiologist to provide a free text description of the reason. Examination and Request Data may be filled automatically by the RI using digital imaging and communications in medicine (DICOM) data for the current examination, and the reason for the request may, in some examples, be recorded manually by the radiologist using the RI. The RI interface may be integrated into systems used by the radiologist to view imaging studies and to prepare radiology reports.

The relevance determination system 225 determines the relevance of examinations to one another based on internal references in the examination reports. The relevance determination system 225 may include a similarity estimator, an examination clustering system, and a group inference system. After all of the radiology and pathology reports are processed, the similarity estimator estimates the similarities between each of the reports for a patient. Given a patient history of n exams, similarity estimator 115 computes a n² relevance matrix between the exams including n(n−1)/2 unique values. Each element in this matrix may contain a value in the range [0,1] (inclusive) indicating the relatedness of the two exams. In a supervised setting, these values may be estimated as scores of a binary relatedness classifier (e.g., logistic regression, neural network, etc.). In an unsupervised setting, these values may be estimated as a similarity score of phrase-vectors which are computed using the noun phrases corresponding to entities extracted in the previous step. Any of these known techniques for determining the similarity between the reports may be used.

For example, the phrases found in a report are converted to a set of vectors. The vectors in the reports are compared for similarities. Based upon these vector comparisons, a similarity score may be determined using the various models discussed above. Note that any report may be similar to a number of different reports, because a patient may have comorbidities.

Next, the examination clustering system takes the similarity results and clusters the reports. This clustering may be done using various machine learning techniques, including, for example, affinity propagation. For example, in another embodiment, when two reports have a similarity score above a specified threshold, the reports may be linked to one another. In another embodiment, clusters of reports may be determined directly via the anatomy labelling that comes out of the anatomy inference process. Any given report may be clustered/linked to a number different reports because a patient has multiple comorbidities. The examination clustering system produces a set of related reports that are similar to one another.

Once clusters have been generated, then the group inference system may infer a group type from anatomy labeling and matching. Such labelling may come from standard dictionaries or databases describing medical conditions, terms, anatomy, etc. For example, Systematized Nomenclature of Medicine (SNOMED), a standardized and multilingual vocabulary of clinical terminology that is used by physicians and other health care providers for the electronic exchange of clinical health information, is one such standard dictionary that may be used for inferring group type from anatomy labeling and matching.

The relevance identification system 230 identifies examinations that are relevant to other examinations with requests for feedback. The radiology feedback system 200 collects requests for feedback generated by the RI system and stores them in a requests database (RDB). For each request in the RDB, the relevance identification system 230 monitors the patient exams subsequent to the request. For each subsequent patient examination, the relevance identification system 230 may compute a matching score between the request and the examination. If a match is found, the requesting radiologist is alerted using the notification and visualization system 235.

In one embodiment, the matching score is computed explicitly as a composite score of anatomy of request matching, evidence of co-reference of the requesting examination in a subsequent examination, and evidence of a subsequent examination being a follow-up of a recommendation in a requesting examination. In another embodiment, the matching score is directly computed based on the content of the subsequent examination and the requesting examination. In some situation where the radiologist requests feedback regarding a report, the link may be fairly direct and identifying the relevance is easy. The matching score may be calculated in various ways using machine learning models or rules based models to calculate the score or values that are combined to calculate the score. In other situations, there may be other reports in the system that are also relevant. Such other reports may be identified because of their similarity to other reports that clearly are relevant. In this way, all the reports that may be relevant to a specific request from the RI system or a recommendation extracted from the radiology reports may be identified and presented to the radiologist to provide feedback.

Once the reports providing feedback are identified, the requesting radiologist may be notified of relevant reports. These notifications may be provided in various ways including emails, text messages, messages using other messaging systems, in the application used by the radiologist to review images and generate reports, etc. The notification and visualization system 235 may display notifications of feedback regarding radiology recommendations or requests. The notification and visualization system 235 may also display the examination related to the recommendation or request along with the matching examination(s). They may be presented individually or side by side on a display. This allows for the radiologist to review both reports to determine whether their initial report was supported by the subsequent matching report. Further, notification and visualization system 235 may provide a mechanism for the requesting radiologist to accept or reject the feedback based on the relevance of the feedback. In some embodiments, the request is not removed from the RDB until the radiologist accepts or acknowledges the feedback.

The model update system 240 may update the recommendation based identification system 220, the relevance determination system 225, and the relevance identification system 230 based on the acceptance or rejection of a suggested relevant examination by the requesting radiologist. This feedback may be used to further train these systems. Various feedback scenarios may exist. For example, the radiology examination identifies the possibility of a malignant growth and recommends a biopsy. The biopsy is performed, but the pathology comes back negative. This feedback to the radiologist indicates a false positive, because the radiologist indicated a malignant growth, but the pathology report indicated that the growth was not malignant. As pathology reports are typically considered ground truth, they may be used to provide this feedback to the radiologist. It is noted that a false negative situation will not provide direct feedback but may result in feedback when later reports are identified as relevant, at which time feedback may be provided to the radiologist indicating that the initial report incorrectly reported not malignant growth. Such feedback may be valuable in helping radiologists to improve their diagnostic skills by learning from misdiagnoses.

The radiology feedback system provides various benefits including continuous feedback that allows the radiologist to improve and to identify misdiagnoses. The radiology feedback system may also be used in peer review and peer learning. The peer review may be used to determine which radiologists are performing well or those that might need further training. In current radiology clinical workflows, the radiologist does not typically receive feedback regarding their examinations because the various data needed to provide such feedback is found in various locations. Further, there is no mechanism to analyze the large number of radiology and pathology reports to identity relevant reports that may provide useful feedback to the radiologist. This technical problem is solved by the radiology feedback system. The radiology feedback system analyzes radiology reports to identify recommendations for further actions based upon the radiology examination. Moreover, the radiology feedback system allows for the radiologist to explicitly call for feedback. The radiology feedback system also analyzes the radiology and pathology (and possibly other related reports) to determine which reports are relevant to one another. This relevance information may then be used to identify subsequent or prior reports that are relevant to the current radiology report. These relevant reports are then presented to the radiologist to provide feedback. The radiologist can use this feedback to verify their original diagnosis. This allows for the radiologist to use such feedback to improve their diagnosis skills. By overcoming the technological problem of not providing feedback to the radiologists these various benefits may be achieved.

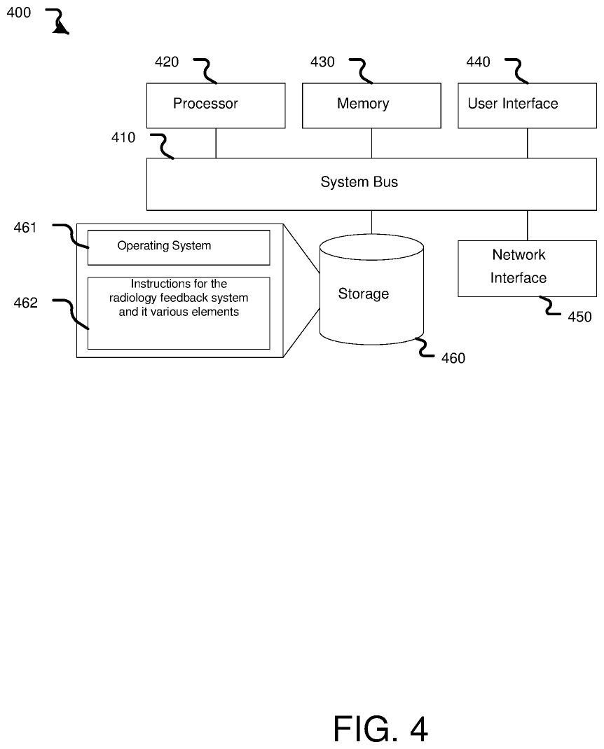

FIG. 4 illustrates an exemplary hardware device 400 for implementing the radiology feedback system 200. This device may implement the whole radiology feedback system 200, or may also implement various elements of it such as the automated report processing system, anatomy inference system 215, recommendation identification system 220, relevance determination system 225, relevance identification system 230, notification visualization system 235, and model update system 240. As shown, the device 400 includes a processor 420, memory 430, user interface 440, network interface 450, and storage 460 interconnected via one or more system buses 410. It will be understood that FIG. 4 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, a graphics processing unit (GPU), field programmable gate array (FPGA), application-specific integrated circuit (ASIC), any processor capable of parallel computing, or other similar devices. The processor may also be a special processor that implements machine learning models.

The memory 430 may include various memories such as, for example L1, L2, or L3 cache or system memory. As such, the memory 430 may include static random-access memory (SRAM), dynamic RAM (DRAM), flash memory, read only memory (ROM), or other similar memory devices.

The user interface 440 may include one or more devices for enabling communication with a user and may present information to users. For example, the user interface 440 may include a display, a touch interface, a mouse, and/or a keyboard for receiving user commands. In some embodiments, the user interface 440 may include a command line interface or graphical user interface that may be presented to a remote terminal via the network interface 450. The user interface 440 may be used to implement the notification and visualization system 235 that presents the notifications and relevant feedback to the radiologists.

The network interface 450 may include one or more devices for enabling communication with other hardware devices. For example, the network interface 450 may include a network interface card (NIC) configured to communicate according to the Ethernet protocol or other communications protocols, including wireless protocols. Additionally, the network interface 450 may implement a TCP/IP stack for communication according to the TCP/IP protocols. Various alternative or additional hardware or configurations for the network interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon which the processor 420 may operate. For example, the storage 460 may store a base operating system 461 for controlling various basic operations of the hardware 400. The storage 462 may store instructions for implementing the radiology feedback system 200 and various elements of the radiology feedback system 200 such as the automated report processing system 210, anatomy inference system 215, recommendation identification system 220, relevance determination system 225, relevance identification system 230, notification visualization system 235, and model update system 240.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered to be “non-transitory machine-readable media.” As used herein, the term “non-transitory” will be understood to exclude transitory signals but to include all forms of storage, including both volatile and non-volatile memories.

While the system 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Such plurality of processors may be of the same or different types. Further, where the device 400 is implemented in a cloud computing system, the various hardware components may belong to separate physical systems. For example, the processor 420 may include a first processor in a first server and a second processor in a second server.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method for managing feedback for a radiologist, the method comprising:
receiving a feedback request associated with an exam, the feedback request comprising one or more of exam data, request identification data, or information indicating a reason for the feedback request;
storing the feedback request in a database;
monitoring one or more medical information systems for a subsequent patient exam, the subsequent patient exam corresponding to the feedback request based on one of the exam data or the request identification data;
generating a subsequent exam alert in response to identifying the subsequent patient exam; and
receiving an acceptance or a rejection of feedback related to the feedback request.

2. The method of claim 1, wherein the feedback request is a request from the radiologist to provide feedback related to a radiology report for the exam.

3. The method of claim 2, wherein the feedback request is extracted from the radiology report using a language processing model and fields of the feedback request are prefilled by the natural language processing model.

4. The method of claim 2, further comprising:
determining relevance of a plurality of medical reports to one another based upon one or more of anatomy identified in the radiology report, reference to the radiology report in the identified subsequent examination, or an indication that the subsequent examination is a follow-up of a recommendation in the radiology report; and
identifying a medical report related to the feedback based upon the determined relevance of the plurality of medical reports to one another.

5. The method of claim 4, wherein determining relevance of the plurality of medical reports is further based upon extracted patient medical information, the method further comprising:
clustering similar medical reports; and
inferring a group type for the clustered medical reports and labeling the clustered medical reports with the inferred group type.

6. The method of claim 4, wherein the plurality of medical reports includes radiology reports and pathology reports.

7. The method of claim 4, wherein processing the plurality of patient medical reports includes document structure processing, syntactic parsing of an output of the document structure processing, extracting entities from an output of the syntactic parsing, and determining an anatomy inference on the extracted entities.

8. The method of claim 1, further comprising receiving an acceptance or a rejection response from the radiologist and updating a model configured to identify a medical report related to the feedback request.

9. The method of claim 8, further comprising deleting the feedback request after receiving the acceptance response.

10. The method of claim 1, wherein the medical information systems comprise one or more of a radiology information system (RIS), laboratory information system (LIS), picture archiving and communication system (PACS), or an electronic medical record (EMR).

11. A device for providing feedback to a radiologist, comprising:
a memory;
a processor coupled to the memory, wherein the processor is further configured to:
receive a plurality of medical reports;
process the plurality of medical reports to produce a processed report that extracts patient medical information;
receive a feedback request related to a radiology report;
identify a medical report related to the feedback request; and
provide a notification to the radiologist regarding the identified medical report related to the feedback request.

12. The device of claim 11, further comprises visualizing the identified medical report and the radiology report for the radiologist.

13. The device of claim 11, wherein the feedback request is a request from the radiologist to provide feedback related to the radiology report.

14. The device of claim 11, wherein the feedback request is extracted from the radiology report using a language processing model.

15. The device of claim 11, further comprising determining the relevance of the plurality of medical reports to one another and wherein identifying a medical report related to the feedback request is based upon the determined relevance of the plurality of medical reports to one another.

16. The device of claim 11, further comprising receiving an acceptance/rejection response from the radiologist and updating a model configured to identify a medical report related to the feedback request.

17. The device of claim 11, wherein identifying a medical report related to the feedback request includes calculating a score based upon one of anatomy identified in the radiology report, reference to the radiology report in a subsequent examination, and an indication that a subsequent examination is a follow-up of a recommendation in the radiology report.

18. The device of claim 11, further comprising:
estimating the similarity between the plurality of medical reports based upon the extracted patient medical information;
clustering similar medical reports; and
inferring a group type for the clustered medical reports and labeling the clustered medical reports with the inferred group type.

19. The device of claim 11, wherein processing the plurality of patient medical reports includes document structure processing, syntactic parsing of an output of the document structure processing, extracting entities from an output of the syntactic parsing, and determining an anatomy inference on the extracted entities.

20. The device of claim 11, wherein the plurality of medical reports includes radiology reports and pathology reports.
